(19) United States
(12) Patent Application Publication
(10) Pub. No.: US 2022/0000497 A1
(43) Pub. Date: Jan. 6, 2022

(54) GUIDING DEVICE AND ULTRASOUND DEVICE SYSTEM

(71) Applicant: OLYMPUS CORPORATION, Tokyo (JP)

(72) Inventors: Yasuyuki MATSUMURA, Hachioji-shi (JP); Takamitsu SAKAMOTO, Hachioji-shi (JP); Ken FUJISAKI, Sagamihara-shi (JP)

(73) Assignee: OLYMPUS CORPORATION, Tokyo (JP)

(21) Appl. No.: 17/478,360

(22) Filed: Sep. 17, 2021

Related U.S. Application Data
(63) Continuation of application No. PCT/JP2019/012981, filed on Mar. 26, 2019.

(51) Int. Cl. A61B 17/17; A61B 17/32

(52) U.S. Cl. CPC A61B 17/1732; A61B 2017/320082; A61B 17/320068

(57) ABSTRACT

A guiding device includes: a guide having a tubular shape. The guide can include a first through hole for insertion of an ultrasound probe. The guide can be configured to guide movement of the ultrasound probe inserted through the first through hole. A protrusion can protrude from one end of the guide and can have a sectional shape that corresponds to a sectional shape of the distal portion of the ultrasound probe, the sectional shape of the protrusion being orthogonal to an axis of the first through hole, and the sectional shape of the distal portion of the ultrasound probe being orthogonal to the axis of the first through hole.

This application is a continuation of International Application No. PCT/JP2019/012981, filed on Mar. 26, 2019, the entire contents of which are incorporated herein by reference.

### BACKGROUND

The present disclosure relates to a guiding device and an ultrasound device system.

It has been known that, in anterior cruciate ligament reconstruction with a bone tendon bone (BTB) technique for fixing a tendon to which a rectangular bone chip is attached, a rectangular bone tunnel for fixing the bone chip is formed, by an ultrasound probe, at the portion to which the anterior cruciate ligament adheres on the side face of a lateral femoral condyle (e.g., refer to WO 2018/078831 A). At the time of formation of such a rectangular bone tunnel by an ultrasound probe, because of a narrow articular cavity, two quadrangular bone tunnels smaller than a desired rectangular bone tunnel are first formed adjacently and then the two bone tunnels are linked together, resulting in one bone tunnel.

### SUMMARY

According to one aspect of the present disclosure, there is provided a guiding device including: a guide having a tubular shape, the guide including a first through hole for insertion of an ultrasound probe that forms, by using ultrasound vibration, a tunnel to a bone with a distal portion of the ultrasound probe in contact with the bone, the guide being configured to guide movement of the ultrasound probe inserted through the first through hole; and a protrusion protruding from one end of the guide, the protrusion having a sectional shape identical to a sectional shape of the distal portion of the ultrasound probe, the sectional shape of the protrusion being orthogonal to an axis of the first through hole, and the sectional shape of the distal portion of the ultrasound probe being orthogonal to the axis of the first through hole.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 illustrates a schematic configuration of an ultrasound device system according to an exemplary embodiment;

FIG. 2 is a perspective view of the external shape of a distal treatment tool of an ultrasound probe according to an exemplary embodiment;

FIG. 3 illustrates a condition of formation of a bone tunnel by an ultrasound device;

FIG. 4 is a perspective view of the external appearance of a guiding device according to an exemplary embodiment;

FIG. 5 is a sectional view of the guiding device according to an exemplary embodiment;

FIG. 6 illustrates a state where a miniature drill is stuck in a treatment target region;

FIG. 7 illustrates a state where the miniature drill is inserted through a second through hole;

FIG. 8 illustrates a state of formation of a first bone tunnel to the treatment target region by the distal treatment tool;

FIG. 9 illustrates a state where a protrusion is inserted in the first bone tunnel;

FIG. 10 illustrates a state of formation of a second bone tunnel to the treatment target region by the distal processing tool;

FIG. 11 illustrates the treatment target region having the first bone tunnel and the second bone tunnel;

FIG. 12 illustrates the treatment target region having a desired rectangular bone tunnel;

FIG. 13 illustrates part of an ultrasound device provided with a guiding device according to an exemplary embodiment; and

FIG. 14 illustrates an ultrasound probe having entered inside a treatment target region.

### DETAILED DESCRIPTION

Embodiments of a guiding device will be described below. Note that the present disclosure is not limited to the embodiments.

### First Embodiment

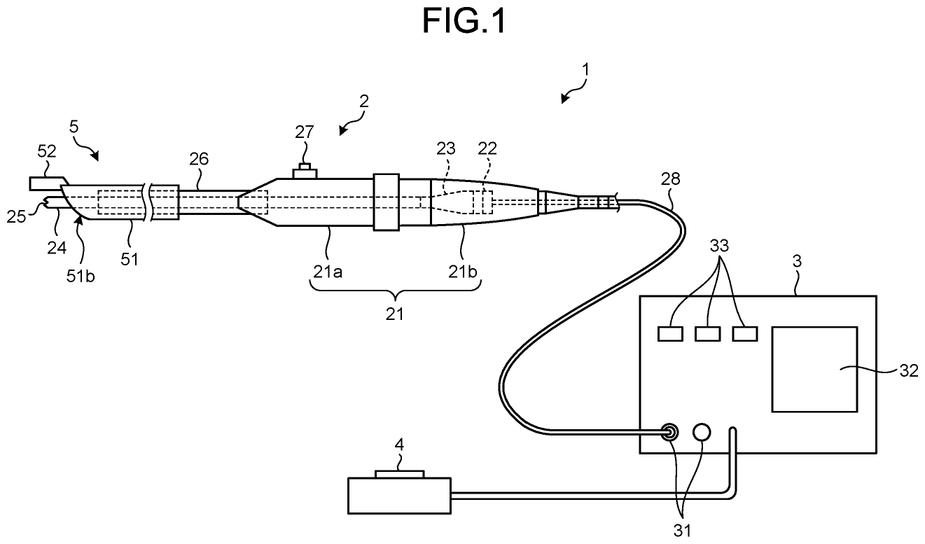

FIG. 1 illustrates a schematic configuration of an ultrasound device system 1 according to a first embodiment. The ultrasound device system 1 according to the first embodiment includes an ultrasound device 2, a power unit 3, and a foot switch 4. The ultrasound device 2 and the power unit 3 are connected through a cable 28, and thus the power unit 3 performs supply of drive power or communication of a control signal to the ultrasound device 2 through the cable 28. The power unit 3 is provided with a plurality of connectors 31 for connection of the cable 28 or the like, a plurality of operating switches 33, and a display screen 32 that displays information necessary for treatment.

The ultrasound device 2 includes a device main body 21 and an ultrasound probe 24. The device main body 21 includes a housing 21a through which the ultrasound probe 24 penetrates and an ultrasound generator 21b detachably attached to the housing 21a. An ultrasound transducer 22 including a piezoelectric body and a horn 23 for transmitting ultrasound efficiently are housed inside the ultrasound generator 21b. With the ultrasound generator 21b attached to the housing 21a, ultrasound vibration generated by the ultrasound generator 21b is transmitted to a distal treatment tool 25 of the ultrasound probe 24 with the proximal end side of the ultrasound probe 24 and the distal end side of the horn 23 in connection. The upper face of the housing 21a is provided with an operating switch 27 that gives an instruction for turning ultrasound vibration on or off in response to an operation due to a finger. The foot switch 4 has a function similar to the function of the operating switch 27, and gives an instruction for turning ultrasound vibration on or off in response to an operation due to a foot. The outer circumference of the ultrasound probe 24 is partially covered with a sheath 26 by an arbitrary length from the housing 21a.

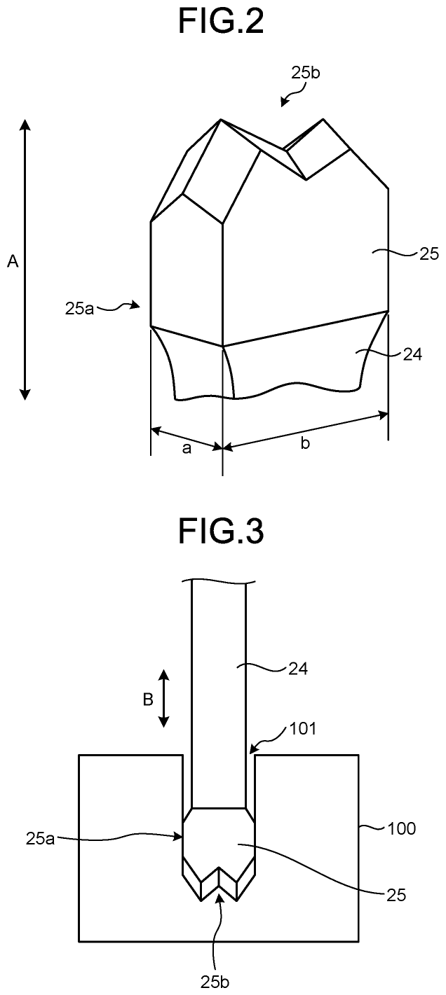

FIG. 2 is a perspective view of the external shape of the distal treatment tool 25 of the ultrasound probe 24 according to the first embodiment. The ultrasound probe 24 according to the first embodiment is integrally formed with the distal treatment tool 25 provided on the distal end side in the direction of an arrow A in the figure corresponding to the axial direction of the ultrasound probe 24. Examples of the material of the ultrasound probe 24 and the distal treatment tool 25 that may be used include titanium alloys. The distal treatment tool 25 serves as an excision tool with ultrasound vibration, and includes a base 25a and a distal portion 25b. The base 25a has a shape that defines the contour shape of a bone tunnel that the ultrasound probe 24 forms to a bone. The sectional shape orthogonal to the axis of the base 25a is rectangular, having two sides being each a in length and the other two sides being each b (<a) in length. The distal portion 25b has a mountain shape for contact with a bone in the travel direction of the ultrasound probe 24.

Next, formation of a bone tunnel 101 by the ultrasound probe 24 will be described with reference to FIG. 3. With the distal treatment tool 25 of the ultrasound probe 24 in contact with a treatment target region 100 of bone, ultrasound vibration in the direction of an arrow B in the figure causes hammering effect, so that a part of bone mechanically hit with the distal treatment tool 25 is crushed into minute granules. Then, a surgical operator pushes the distal treatment tool 25 into the treatment target region 100, so that the distal treatment tool 25 enters inside the treatment target region 100 while crushing the bone. Thus, formed is the bone tunnel 101 in a rectangular shape identical to the sectional shape orthogonal to the axis of the base 25a.

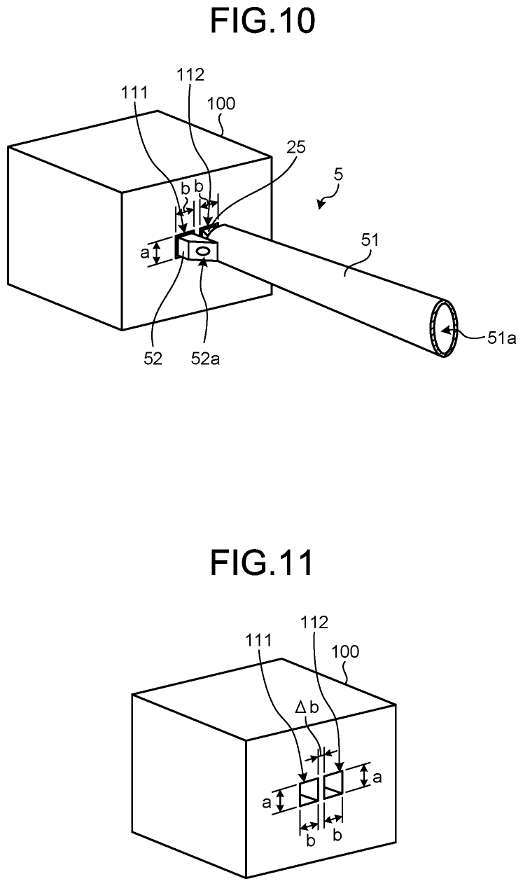

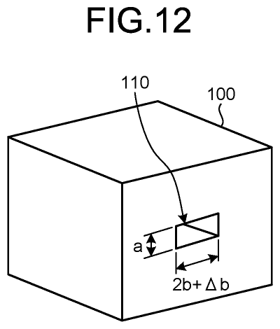

In anterior cruciate ligament reconstruction with the ultrasound device system 1 according to the first embodiment, a first bone tunnel 111 and a second bone tunnel 112 each in a rectangular shape being a long by b broad, to be described later, are formed adjacently at a certain interval Δb (<b) at the portion to which the anterior cruciate ligament adheres on the side face of a lateral femoral condyle as the treatment target region 100 (refer to FIG. 11). Then, linking the first bone tunnel 111 and the second bone tunnel 112 together results in final formation of a bone tunnel 110 in a rectangular shape having two sides being each a in length and the other two sides being each 2b+Δb in length (refer to FIG. 12).

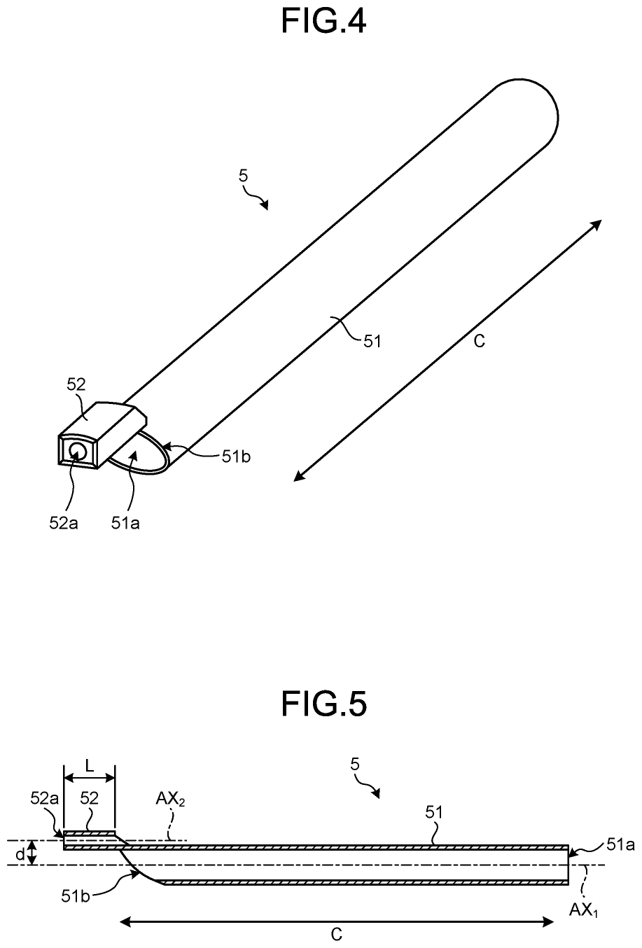

Next, a guiding device 5 for guiding the ultrasound probe 24 in the travel direction will be described. FIG. 4 is a perspective view of the external appearance of the guiding device 5 according to the first embodiment. FIG. 5 is a sectional view of the guiding device 5 according to the first embodiment.

The guiding device 5 according to the first embodiment includes a guide 51 and a protrusion 52. The guide 51 is tubular in shape, having a first through hole 51a for insertion of the ultrasound probe 24. The guide 51 regulates travel of the ultrasound probe 24 in a certain direction and guides the ultrasound probe 24 in the travel direction. The guide 51 has a cut-away portion 51b cutaway obliquely across the direction of an arrow C in the figure corresponding to the axial direction of the guide 51. Thus, the guide 51 has space near the protrusion 52, so that an improvement may be made in the visibility of the distal treatment tool 25 of the ultrasound probe 24 inserted through the first through hole 51a.

The protrusion 52 protrudes by a length L and has a second through hole 52a for insertion of a miniature drill 6, parallel to the first through hole 51a. Note that, in the first embodiment, the term “miniature” means the diameter of a circle smaller than the sectional shape orthogonal to the axis of the ultrasound probe 24. In the first embodiment, the diameter of the miniature drill 6 is 2.4 [mm]. The inter-axis distance d between the axis AX₁ of the first through hole 51a and the axis AX₂ of the second through hole 52a illustrated in FIG. 5 is shorter than the longer side of a completed rectangular bone tunnel 110 (refer to FIG. 12). The protrusion 52 is provided on the outer circumferential face on the distal end side as one end side of the guide 51. The distal end of the protrusion 52 is closer to the distal end side in the axial direction than the distal end of the guide 51 is. The proximal end of the protrusion 52 is located on the outer circumferential face on the distal end side of the guide 51. The sectional shape orthogonal to the axis AX₂ of the protrusion 52 is non-circular and is identical to the sectional shape orthogonal to the axis of the distal treatment tool 25. Preferably, the sectional shape orthogonal to the axis AX₂ of the protrusion 52 is smaller in size than the sectional shape orthogonal to the axis of the distal treatment tool 25, so that the protrusion 52 fits in a bone tunnel formed by the distal treatment tool 25. The length L of the protrusion 52 is set such that the guide 51 is prevented from deviating in position in a case where the protrusion 52 is put into the bone tunnel. For example, the length L is set at 5 [mm].

Due to insertion of the ultrasound probe 24 into the first through hole 51a of the guiding device 5, the ultrasound probe 24 is guided along the inner circumferential face of the first through hole 51a, so that the travel direction of the ultrasound probe 24 is determined. The distal treatment tool 25 protruding from the opening on the distal end side in the axial direction of the first through hole 51a, due to insertion of the ultrasound probe 24 through the first through hole 51a of the guide 51, is pressed against the treatment target region 100.

Next, described will be a procedure of final formation of a desired rectangular bone tunnel to the treatment target region 100 of bone with the ultrasound probe 24 guided by the guiding device 5.

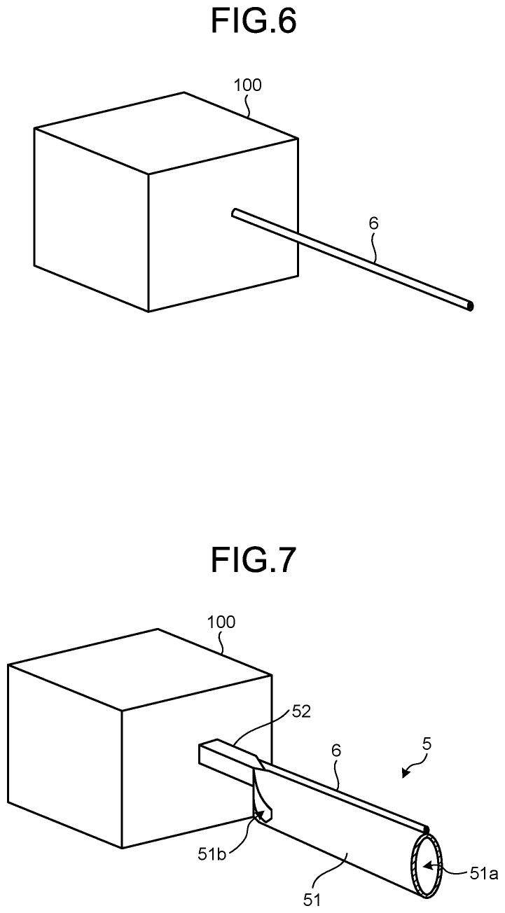

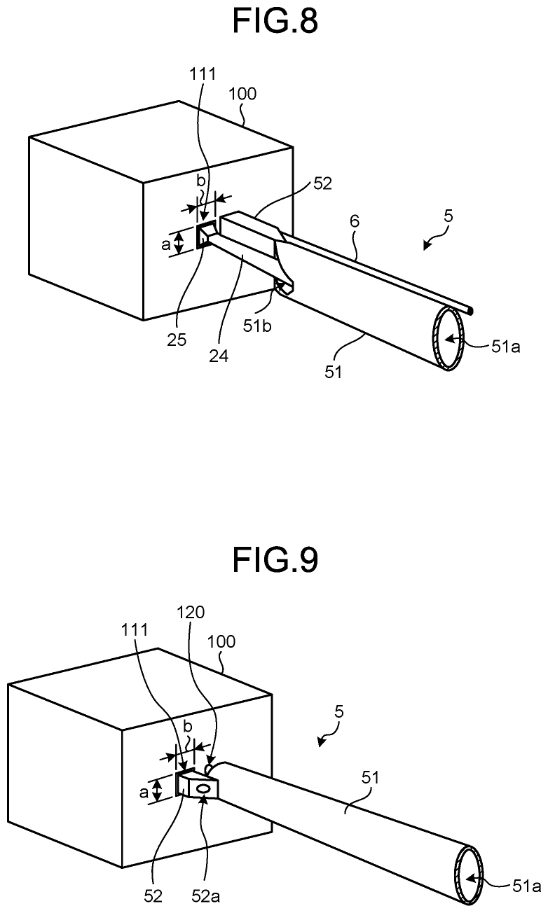

FIG. 6 illustrates a state where the miniature drill 6 is stuck in the treatment target region 100 of bone. Note that the treatment target region 100 is rendered by modeling of a region of bone as a treatment target to which the ultrasound probe 24 forms a bone tunnel. First, the surgical operator makes the axis of the miniature drill 6 identical to the direction in which a bone tunnel is to be formed to the treatment target region 100, and sticks, as illustrated in FIG. 6, the miniature drill 6 into the treatment target region 100. Next, as illustrated in FIG. 7, the surgical operator inserts the miniature drill 6 stuck in the treatment target region 100 through the second through hole 52a of the protrusion 52 in the guiding device 5 such that the protrusion 52 has contact with the surface of the treatment target region 100. Next, the surgical operator inserts the ultrasound probe 24 through the first through hole 51a of the guide 51 in the guiding device 5 such that the distal treatment tool 25 protrudes from the cut-away portion 51b. Then, as illustrated in FIG. 8, the surgical operator makes one side having a length of a in the sectional shape orthogonal to the axis of the distal treatment tool 25 and the face opposed to the ultrasound probe 24 of the protrusion 52 in parallel, and makes the distal treatment tool 25 in contact with the surface of the treatment target region 100. In this case, the ultrasound probe 24 guided along the inner circumferential face of the first through hole 51a travels in the direction parallel to the axis of the miniature drill 6, namely, in the direction in which a bone tunnel is to be formed to the treatment target region 100. Then, the surgical operator brings the ultrasound probe 24 into ultrasound vibration, so that, as illustrated in FIG. 8, a first bone tunnel 111 having two sides being each a in length and the other two sides being each b in length is formed to the treatment target region 100 by the distal treatment tool 25.

Next, the surgical operator removes the miniature drill 6 and the ultrasound probe 24 from the treatment target region 100. After that, as illustrated in FIG. 9, the surgical operator rotates the orientation of the guiding device 5 counterclockwise by 180 degrees, and inserts the protrusion 52 into the first bone tunnel 111. Thus, the distal end of the guide 51 is opposed in location to a drilled hole 120 made in the treatment target region 100 by the miniature drill 6, resulting in positioning of the guiding device 5 to the treatment target region 100. Next, as illustrated in FIG. 10, the surgical operator inserts the ultrasound probe 24 through the first through hole 51a of the guide 51 such that the distal treatment tool 25 protrudes from the first through hole 51a. The surgical operator makes the one side of the distal treatment tool 25 and the face opposed to the ultrasound probe 24 of the protrusion 52 in parallel, and makes the distal treatment tool 25 in contact with the surface of the treatment target region 100. In this case, the travel direction of the ultrasound probe 24 is regulated in the direction in which a bone tunnel is to be formed to the treatment target region 100, along the inner circumferential face of the first through hole 51a. Then, as illustrated in FIGS. 10 and 11, the surgical operator brings the ultrasound probe 24 into ultrasound vibration, so that a second bone tunnel 112 having two sides being each a in length and the other two sides being each b in length is formed to the treatment target region 100 by the distal treatment tool 25. Such formation of the second bone tunnel 112 to the treatment target region 100 as above leads to the direction of penetration of the second bone tunnel 112 parallel to the direction of penetration of the first bone tunnel 111.

After the formation of the second bone tunnel 112 to the treatment target region 100, the surgical operator removes the ultrasound probe 24 and the guiding device 5 from the treatment target region 100. Thus, as illustrated in FIG. 11, the treatment target region 100 has the first bone tunnel 111 and the second bone tunnel 112 adjacent to each other with a bone portion having a certain thickness Δb between the first bone tunnel 111 and the second bone tunnel 112. The thickness Δb satisfies the following expression: Δb<b, preferably, the following expression: Δb<0.5b. Then, with a dilator or the like, the surgical operator removes the bone portion between the first bone tunnel 111 and the second bone tunnel 112 in the treatment target region 100 such that the first bone tunnel 111 and the second bone tunnel 112 are in communication, as illustrated in FIG. 12, resulting in final formation of a rectangular bone tunnel 110 that is a long by (2b+Δb) broad. As an example, in a case where the following expressions are satisfied: a=5 [mm], b=4 [mm], and Δb=2 [mm], formed is a rectangular bone tunnel 110 having two sides being each 5 [mm] and the other two sides being each 10 [mm].

In the first embodiment, guiding the ultrasound probe 24 with the guiding device 5 enables adjacent formation of the first bone tunnel 111 and the second bone tunnel 112 at targeted positions and angles to the treatment target region 100. Thus, regardless of any surgical operator, a desired rectangular bone tunnel 110 may be accurately formed in the direction in which a bone tunnel is to be formed. In particular, the effect is more salient to surgical operators low in the level of skill.

In the first embodiment, the inter-axis distance d between the axis AX₁ of the first through hole 51a and the axis AX₂ of the second through hole 52a illustrated in FIG. 5 with the length of the protrusion 52 orthogonal to the axis AX₂ of the second through hole 52a defined as b is shorter than the length (2b+Δb) of the longer side of the rectangular bone tunnel 110 (b<d<2b+Δb). As described above, because the thickness Δb satisfies the following expression: Δb<b, preferably, the following expression: Δb<0.5b, the inter-axis distance d satisfies the following expression: b<d<3b, preferably, the following expression: b<d<2.5b. Thus, at the time of formation of the second bone tunnel 112 to the treatment target region 100, the distal treatment tool 25 in contact with the surface of the treatment target region 100 is located on the drilled hole 120 made by the miniature drill 6 inserted through the second through hole 52a. Therefore, formation of the second bone tunnel 112 to the treatment target region 100 leads to elimination of the drilled hole 120 from the treatment target region 100, so that such a useless hole may be prevented from remaining in the treatment target region 100.

### Second Embodiment

Next, a second embodiment will be described. In the following description, constituents in the second embodiment similar to those in the first embodiment described above are denoted with the same reference signs, and the descriptions thereof will be omitted. A procedure of formation of a desired rectangular bone tunnel 110 to a treatment target region 100 with an ultrasound probe 24 guided by a guiding device 5A is substantially similar to that in the first embodiment, and thus the detailed descriptions of steps the same as those in the first embodiment will be omitted.

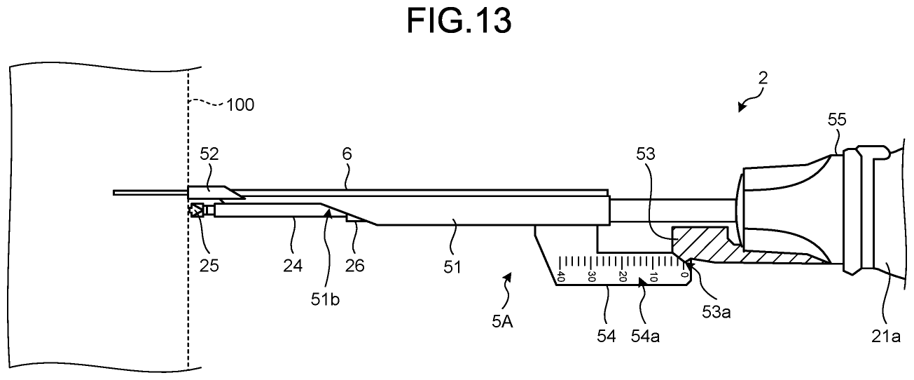

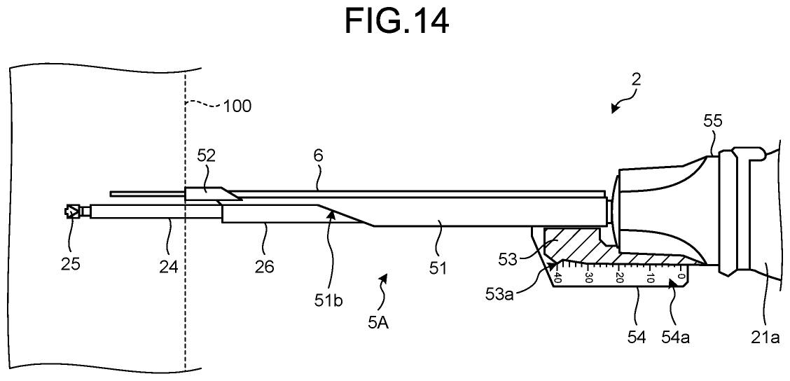

FIG. 13 illustrates part of an ultrasound device 2 provided with the guiding device 5A according to the second embodiment. FIG. 14 illustrates the ultrasound probe 24 having entered inside the treatment target region 100.

As illustrated in FIG. 13, the guiding device 5A according to the second embodiment has a rib or a blade 54 having a scale 54a, provided on the outer circumferential face on the rear end side in the axial direction of a guide 51. The blade 54 tabular in shape erects on the outer circumferential face of the guide 51 and extends in the axial direction of the guide 51. The scale 54a indicates the entry distance at the time of entry of a distal treatment tool 25 of the ultrasound probe 24 to the treatment target region 100 along with formation of a bone tunnel. The blade 54 is located opposite to a protrusion 52 in the direction orthogonal to the axis AX₁ of the guide 51. The guiding device 5A includes a cap 55 attachable to the distal portion of a housing 21a of the ultrasound device 2. The cap 55 is provided with a regulating plate 53 as a regulator that regulates, due to contact with the blade 54 around the axis of the guide 51, rotation of the guide 51 around a miniature drill 6 inserted through a second through hole 52a.

Regarding the guiding device 5A according to the second embodiment, at the time of formation of a first bone tunnel 111 to the treatment target region 100, the ultrasound probe 24 is inserted through a first through hole 51a with regulation of rotation of the guide 51 with the blade 54 and the regulating plate 53 in contact. Thus, performed may be relative positioning of the distal treatment tool 25 to the miniature drill 6 inserted through the second through hole 52a. As a result, enhanced may be the accuracy of position at the time of formation of the first bone tunnel 111 to the treatment target region 100 by the distal treatment tool 25.

The regulating plate 53 is provided with a pointer 53a in a mountain shape as an indicator that indicates the position on the scale 54a corresponding to the entry distance of the distal treatment tool 25 with the regulating plate 53 and the blade 54 in contact. As illustrated in FIG. 13, with the distal treatment tool 25 in contact with the surface of the treatment target region 100, the pointer 53a indicates 0 [mm] on the scale 54a. As illustrated in FIG. 14, with the distal treatment tool 25 having entered inside the treatment target region 100, due to relative movement of the blade 54 to the regulating plate 53, for example, the pointer 53a indicates 40 [mm] on the scale 54a as the entry distance of the distal treatment tool 25.

Regarding the guiding device 5A according to the second embodiment, a surgical operator may read the entry distance of the distal treatment tool 25 from the scale 54a during formation of the first bone tunnel 111 and a second bone tunnel 112 to the treatment target region 100 with the ultrasound probe 24. Thus, regardless of any surgical operator, the first bone tunnel 111 and the second bone tunnel 112 each having a targeted depth may be easily formed.

As above, the guiding device is effective in formation of a desired rectangular bone tunnel in the direction in which a bone tunnel is to be formed, with an ultrasound probe.

The guiding device according to the present disclosure enables, as an effect, assistance to formation of a desired bone tunnel in the direction in which a bone tunnel is to be formed, with an ultrasound probe.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general concept as defined by the appended claims and their equivalents.

What is claimed is:

1. A guiding device comprising:
a guide having a tubular shape, the guide including a first through hole for insertion of an ultrasound probe that is configured to transmit ultrasound vibration to form a tunnel in a bone using a distal portion of the ultrasound probe, the guide being configured to guide movement of the ultrasound probe inserted through the first through hole; and
a protrusion protruding from one end of the guide, the protrusion having a sectional shape corresponding to a sectional shape of the distal portion of the ultrasound probe, the sectional shape of the protrusion being orthogonal to an axis of the first through hole, and the sectional shape of the distal portion of the ultrasound probe being orthogonal to the axis of the first through hole.

2. The guiding device according to claim 1, wherein the protrusion includes a second through hole configured to receive a miniature drill, the second through hole extending parallel to the first through hole.

3. The guiding device according to claim 1, wherein the guide includes a distal portion cutaway from the one end of the guide obliquely across an axis of the guide.

4. The guiding device according to claim 1, wherein the protrusion includes a proximal end on an outer circumferential face of the guide closer to the one end of the guide.

5. The guiding device according to claim 1, further comprising:
a rib extending from an outer circumferential face of the guide, wherein
the rib includes a scale that extends in an axial direction of the guide and indicates an entry distance of the distal portion of the ultrasound probe into a bone tunnel.

6. An ultrasound device system comprising:
an ultrasound device including an ultrasound probe configured to transmit ultrasound vibration to form a tunnel in a bone with a distal portion of the ultrasound probe; and
a guiding device including:
a guide having a tubular shape and including a first through hole configured to receive the ultrasound probe, the guide being configured to guide movement of the ultrasound probe; and
a protrusion protruding from one end of the guide, the protrusion having a sectional shape corresponding to a sectional shape of the distal portion of the ultrasound probe, the sectional shape of the protrusion being orthogonal to an axis of the first through hole, and the sectional shape of the distal portion of the ultrasound probe being orthogonal to the axis of the first through hole.
